(19) United States
(12) Patent Application Publication
(10) Pub. No.: US 2025/0188409 A1
(43) Pub. Date: Jun. 12, 2025

(54) NANO-MATERIAL FOR GENERATING ATP AND NADPH UNDER RED LIGHT EXCITATION AND PREPARATION METHOD THEREOF

(71) Applicant: ZHEJIANG UNIVERSITY, Hangzhou (CN)

(72) Inventors: Pengfei CHEN, Hangzhou (CN); Chenhui GU, Hangzhou (CN); Xiangfeng LIN, Hangzhou (CN)

(21) Appl. No.: 19/060,731

(22) Filed: Feb. 23, 2025

Related U.S. Application Data
(63) Continuation of application No. PCT/CN2022/139149, filed on Dec. 14, 2022.

(30) Foreign Application Priority Data
Aug. 22, 2022 (CN) 202211004743.7

(51) Int. Cl. C12N 5/04; C12N 13/00

(52) U.S. Cl. CPC C12N 5/04; C12N 13/00

(57) ABSTRACT

Disclosed are a nano-material for generating ATP and NADPH under red light excitation and a preparation method thereof. By I extracting and nanocrystallizing a plant-derived thylakoid, a nano-thylakoid is obtained, and ATP and NADPH are generated, which are respectively used to enhance biological energy metabolism and substance metabolism, maintain cellular homeostasis, and realize a variety of engineering designs, thus providing a new thought for the preparation and application of biological energy and substance metabolism regulation materials.

### CROSS-REFERENCE TO RELATED APPLICATIONS

This application is a continuation of International Patent Application No. PCT/CN2022/139149 with a filing date of Dec. 14, 2022, designating the United States, now pending, and further claims priority to Chinese Patent Application No. 202211004743.7 with a filing date of Aug. 22, 2022. The content of the aforementioned applications, including any intervening amendments thereto, are incorporated herein by reference.

### TECHNICAL FIELD

The present invention relates to a nano-material, and particularly to a nano-material for generating ATP and NADPH under red light excitation and a preparation method thereof.

### BACKGROUND OF THE PRESENT INVENTION

Biological energy and substance homeostasis are very important for the normal exertion of biological functions, and the anabolism of substances (which are namely a fatty acid, an amino acid, a nucleotide and a steroid) in an organism needs to consume sufficient intracellular energy and reducing equivalent. ATP bears a main transport carrier of energy process in the organism, while NADPH bears a main reducing equivalent of substance synthesis in the organism. Therefore, it is of great significance to develop a nano-material for stably generating ATP and NADPH.

Thylakoid is a main membranous structure in a plant chloroplast, and contains a photosystem I, photosystem II, a Cytb6/f complex and an ATPase complex, which may continuously generate ATP and NADPH under a light condition for autogenous energy metabolism and substance metabolism of plant cells.

At present, an application of a plant thylakoid is mainly limited to the improvement of tumor hypoxia state and the preparation of light absorption materials. The patent with the application number CN202010453892.6 discloses a composite material for simultaneously generating oxygen and active oxygen under near-infrared light excitation, a preparation method therefor and an application thereof, wherein the efficiency of photodynamic therapy for tumors is improved by the characteristic that the thylakoid generates the oxygen and the active oxygen under near-infrared light excitation, but the composite material is only limited to the utilization of thylakoid in oxygen generation, and is not applied to achieve an energy and substance metabolism regulation function of thylakoid. The patent with the application number CN201010136102.8 discloses a preparation method of a light-absorbing nano-material based on a plant thylakoid structure, wherein the light-absorbing nano-material is obtained by mixing a thylakoid with a titanium tetrachloride alcohol solution to roast, but the preparation method is only limited to an optical absorption performance, without paying attention to a biological performance. Such patents are based on a photolysis process of light absorption-oxygen generation of thylakoid, but they are not designed and applied to the ATP and NADPH generation characteristic of thylakoid. Therefore, it is necessary to prepare a nano-material for generating ATP and NADPH under red light excitation and a preparation method thereof, so that the nano-material has the universal effect of generating ATP and NADPH in biological environment, thus fully achieving the regulation effect on the biological energy and substance metabolism.

### SUMMARY OF THE PRESENT INVENTION

Aiming at the defects in the prior art, the present invention provides a nano-material for generating ATP and NADPH under red light excitation and a preparation method thereof. The nano-material may generate ATP and NADPH through a photosystem I, photosystem II, a Cytb6/f complex and an ATPase complex on a thylakoid membrane under red light (˜630 nm) irradiation, which may be respectively used to enhance biological energy metabolism and substance metabolism.

In order to achieve the objective above, the present invention provides the following technical solutions.

A nano-material for generating ATP and NADPH under red light excitation is provided, wherein the nano-material is a nanocrystallized plant thylakoid; and a target particle size of the nanocrystallization is 50 nm to 200 nm.

Preferably, the nanocrystallization is a granulation method, comprising heating, freeze-drying, extrusion and ultrasonic treatment.

Preferably, an ultrasonic extrusion method is used for the nanocrystallization.

Preferably, the particle size of the nanocrystallization is 100 nm.

Preferably, the plant comprises a unicellular plant and a multicellular plant which contain the thylakoid or a similar photosynthetic structure.

Preferably, the plant is a higher multicellular plant.

Preferably, the higher multicellular plant is Spinacia oleracea and Arabidopsis thaliana.

A preparation method of the nano-material for generating ATP and NADPH under red light excitation is provided, which comprises the following steps of: extracting a plant thylakoid, and nanocrystallizing and granulating the plant thylakoid to obtain a nanocrystallized plant thylakoid. Preferably, the plant thylakoid is a Spinacia oleracea leaf thylakoid, and is extracted in the following mode:
in step 1: a Spinacia oleracea leaf material and a buffer A are mixed by a blender; a ratio of the Spinacia oleracea leaf material to the cold buffer A is 1:1 to 1:10 (w/v); ingredients of the buffer A are sorbitol, HEPES-KOH with pH of 7.6, MgCl₂ and 0.1% BSA; and a temperature of the buffer A is 4° C. to 10° C.;
in step 2: the obtained solution in the step 1 is filtered, and a filtrate is centrifuged at 3000 g for 10 minutes;
in step 3: the precipitate in the step 2 is resuspended in a buffer B; and ingredients of the buffer B are sorbitol, HEPES-KOH with pH of 7.6, MgCl₂, EDTA and L-sodium ascorbate; and
in step 4: the product in the step 3 is added into 80/40% Percoll gradient solution; and a part containing a green layer is collected to obtain the thylakoid.

Preferably, the filtration in the second step is implemented by pressing the solution to pass through a piece of fine mesh cotton fabric.

Preferably, a molar ratio of sorbitol, HEPES-KOH and MgCl₂ in the buffer A is 66:10:1.

Preferably, a molar ratio of sorbitol, HEPES-KOH with pH of 7.6, MgCl₂, EDTA and L-sodium ascorbate in the buffer B is 300:50: 5:2: 10.

Preferably, a preparation method of the 80/40% Percoll gradient solution is: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

Preferably, the nanocrystallized granulation is ultrasonic extrusion nanocrystallization, and is specifically carried out in the following mode:
the thylakoid is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

Preferably, conditions of the ultrasonic treatment comprise: a No. 2 amplitude-change pole, 20% to 60% power, turning on for 2 seconds, turning off for 3 seconds, and working for 2 minutes.

Preferably, a pore size of the polycarbonate membrane above is 50 nm to 200 nm.

Preferably, a molar ratio of HEPES-KOH, MgCl₂ and L-sodium ascorbate is 1:1:1.

The present invention has the beneficial effects as follows:
1) According to the thylakoid nano-material provided by the present invention, a relatively uniform nanoscale particle size is achieved, so that the nano-material has broad prospects in cell delivery and biological therapy.
2) According to the thylakoid nano-material provided by the present invention, proteins related to ATP and NADPH generation on the thylakoid are fully retained, and ATP and NADPH may be stably generated under red light excitation.
3) According to the thylakoid nano-material provided by the present invention, there is a long half-life under light and dark conditions, so that continuous working and long-term stability are ensured.
4) According to the thylakoid nano-material provided by the present invention, the nano-material may be encapsulated in lipid vesicle by extrusion for in-vivo delivery, so that the nano-material has a strong engineerable performance.

### DESCRIPTION OF THE DRAWINGS

In order to make the objectives, technical solutions and beneficial effects of the present invention clearer, the present invention provides the following drawings for description:

FIG. 1 shows comparison of particle size distributions of a thylakoid and a nano-thylakoid in Embodiment 1, indicating that a particle size of the thylakoid is about 1 μm, and a particle size of the nano-thylakoid is about 100 nm.

FIG. 2(a) and FIG. 2(b) show transmission electron microscopic characterization of the thylakoid and the nano-thylakoid in Embodiment 1, indicating that the nano-thylakoid remains vesicular.

FIG. 3 shows protein mass spectrometry analysis of the nano-thylakoid in Embodiment 1, indicating the enrichment of proteins related to ATP and NADPH generation.

FIG. 4(a) and FIG. 4(b) show quantitative measurement of ATP and NADPH generation capacities of the nano-thylakoid in Embodiment 1, indicating that ATP and NADPH are significantly generated under a light condition.

FIG. 5(a) and FIG. 5(b) show western blotting of half-lives of key proteins of the nano-thylakoid in Embodiment 1 under light and dark conditions, indicating that the half-lives of the key proteins under the light condition are about 4 hours to 8 hours, and the half-lives of the key proteins under the dark condition are about 3 days to 5 days.

FIG. 6(a) and FIG. 6(b) show quantitative measurement of half-lives of functions of the nano-thylakoid in Embodiment 1 under light and dark conditions, indicating that the half-lives of the functions under the light condition are about 4 hours to 8 hours, and the half-lives of the functions under the dark condition are about 3 days to 5 days.

FIG. 7(a) and FIG. 7(b) show transmission electron microscope characterization of an engineered form of the nano-thylakoid in Embodiment 1 subjected to liposome encapsulation, indicating double-layer vesicle.

### DETAILED DESCRIPTION OF PREFERRED EMBODIMENTS

A nano-material for generating ATP and NADPH under red light excitation and a preparation method thereof provided by the present invention are described in detail hereinafter with reference to embodiments, but the embodiments cannot be understood as limiting the scope of protection of the present invention.

The present invention provides a nano-material for generating ATP and NADPH under red light excitation, wherein the nano-material is a nanocrystallized plant thylakoid. The “nanocrystallization” comprises granulation methods of heating, freeze-drying, extrusion and ultrasonic treatment. Preferably, a granulation method with low physical stimulation is adopted, and more preferably, the extrusion method is adopted for granulation. A target particle size of the “nanocrystallization” is 50 nm to 200 nm. Preferably, the particle size of the nanocrystallization is 100 nm. The “plant” comprises a unicellular plant and a multicellular plant which contain the thylakoid or a similar photosynthetic structure. Preferably, a higher multicellular plant is adopted, and more preferably, Spinacia oleracea, Arabidopsis thaliana, and other plant commonly used in research are adopted. The “thylakoid” comprises a thylakoid in a chloroplast of a plant cell, a thylakoid in a lower plant such as cyanobacteria, and other photosynthetic membrane structure. Preferably, a thylakoid in a chloroplast of a mesophyll cell of a higher plant is adopted, and more preferably, a thylakoid of a Spinacia oleracea leaf is adopted.

The present invention provides a preparation method of the nano-material for generating ATP and NADPH under red light excitation, which comprises the following steps.

In step 1, the plant thylakoid is extracted:
the plant thylakoid, preferably the thylakoid of the Spinacia oleracea leaf, is extracted in the following mode.

A Spinacia oleracea leaf material and a cold buffer A are mixed by a blender in a ratio of 1:1 to 1:10 (w/v). The obtained solution is pressed to pass through a piece of fine mesh cotton fabric, and a filtrate is centrifuged at 3000 g for 10 minutes. A precipitate is gently resuspended in a buffer B. The solution is added to 80/40% Percoll gradient solution. A part containing a green layer is collected to obtain the thylakoid. Ingredients of the buffer A above are 330 mM sorbitol, 50 mM HEPES-KOH with pH of 7.6, 5 mM MgCl₂ and 0.1% BSA. Ingredients of the buffer B above are 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above is: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, the thylakoid is nanocrystallized:
the thylakoid is nanocrystallized by ultrasonic extrusion nanocrystallization, and an ultrasonic extrusion nanocrystallization process is carried out in the following mode.

The thylakoid is subjected to an ultrasonic treatment in a bath ultrasonic instrument, and repeatedly extruded by a polycarbonate porous membrane. Then, the solution is centrifuged at 3000 g for 10 minutes. A precipitate is resuspended in a buffer D. Conditions of the ultrasonic treatment above comprise: a No. 2 amplitude-change pole, 20% to 60% power, turning on for 2 seconds, turning off for 3 seconds, and working for 2 minutes, and preferably 40% power. A pore size of the polycarbonate membrane above is 50 nm to 200 nm, and preferably the pore size is 100 nm. Ingredients of the buffer D above are 10 mM HEPES-KOH, 10 mM MgCl₂ and 10 mM L-sodium ascorbate.

The present invention may also adopt other thylakoid sources, other thylakoid preparation schemes, other nanocrystallization methods, and other target particle sizes of nanocrystallization mentioned above, all of which can achieve the same technical effects.

Embodiment 1. Preparation of 100 nm nano-thylakoid by ultrasonic extrusion nanocrystallization of Spinacia oleracea leaf thylakoid

In step 1, the thylakoid was extracted:
1 g of Spinacia oleracea leaf material and 1 mL of cold buffer A were mixed by a blender. The obtained solution was pressed to pass through a piece of fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain the thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7.6, 5 mM MgCl_{2, 2} mM EDTA and 10 mM L-sodium ascorbate. A preparation method of the 80/40% Percoll gradient solution above was: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, the thylakoid was nanocrystallized:
the thylakoid was subjected to an ultrasonic treatment in a bath ultrasonic instrument, and repeatedly extruded by a polycarbonate porous membrane. Then, the solution was centrifuged at 3000 g for 10 minutes. A precipitate was resuspended in a buffer D. Conditions of the ultrasonic treatment above comprised: a No. 2 amplitude-change pole, 40% power, turning on for 2 seconds, turning off for 3 seconds, and working for 2 minutes. A pore size of the polycarbonate membrane above was 100 nm. Ingredients of the buffer D above were 10 mM HEPES-KOH, 10 mM MgCl₂ and 10 mM L-sodium ascorbate.

### Embodiment 2

### Preparation of 200 nm Nano-Thylakoid By Extrusion Nanocrystallization of Arabidopsis thaliana Leaf Thylakoid

In step 1, the thylakoid was extracted:
1 g of Arabidopsis thaliana leaf material and 5 mL of cold buffer A were mixed by a blender. The obtained solution was pressed to pass through a piece of fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain the thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above was: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, the thylakoid was nanocrystallized:
the thylakoid was repeatedly extruded by a polycarbonate porous membrane. Then, the solution was centrifuged at 3000 g for 10 minutes. A precipitate was resuspended in a buffer D. A pore size of the polycarbonate membrane above was 200 nm. Ingredients of the buffer D above were 10 mM HEPES-KOH, 10 mM MgCl₂ and 10 mM L-sodium ascorbate.

### Embodiment 3

### Preparation of 50 nm Nano-Thylakoid by Ultrasonic Freeze-Drying Nanocrystallization of Tea Leaf Thylakoid

In step 1, the thylakoid was extracted:
1 g of tea leaf material and 10 mL of cold buffer A were mixed by a blender. The obtained solution was pressed to pass through a piece of fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain the thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7.6, 5 mM MgCl_{2, 2} mM EDTA and 10 mM L-sodium ascorbate. A preparation method of the 80/40% Percoll gradient solution above was: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, the thylakoid was nanocrystallized:
the thylakoid was subjected to an ultrasonic treatment in a bath ultrasonic instrument, frozen overnight in a refrigerator at −80° C., freeze-dried by a freeze dryer, and resuspended in a buffer D. Then, the solution was centrifuged at 3000 g for 10 minutes. A precipitate was resuspended in the buffer D. Conditions of the ultrasonic treatment above comprised: a No. 2 amplitude-change pole, 60% power, turning on for 2 seconds, turning off for 3 seconds, and working for 2 minutes. Ingredients of the buffer D above were 10 mM HEPES-KOH, 10 mM MgCl₂ and 10 mM L-sodium ascorbate.

### Comparison of Particle Size Distributions of Thylakoid and Nano-Thylakoid in Embodiment 1

1. The extracted thylakoid and the nanocrystallized thylakoid were resuspended in the buffer and diluted to suitable concentrations, and particle sizes were measured by a Malvin particle size potentiometer.

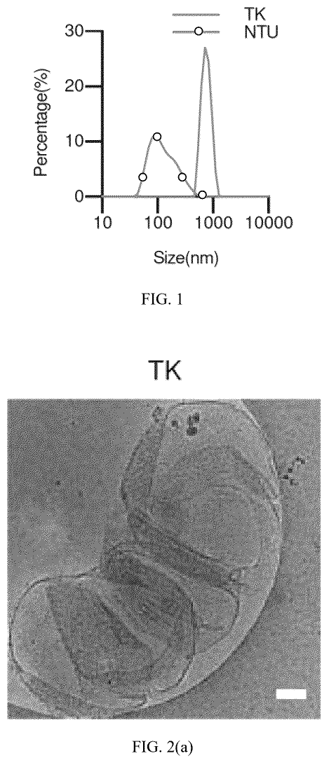

2. According to the measurement, the particle size of the thylakoid was about 1 μm, and the particle size of the nano-thylakoid was about 100 nm, as shown in FIG. 1.

### Transmission Electron Microscopic Characterization of Thylakoid and Nano-Thylakoid in Embodiment 1

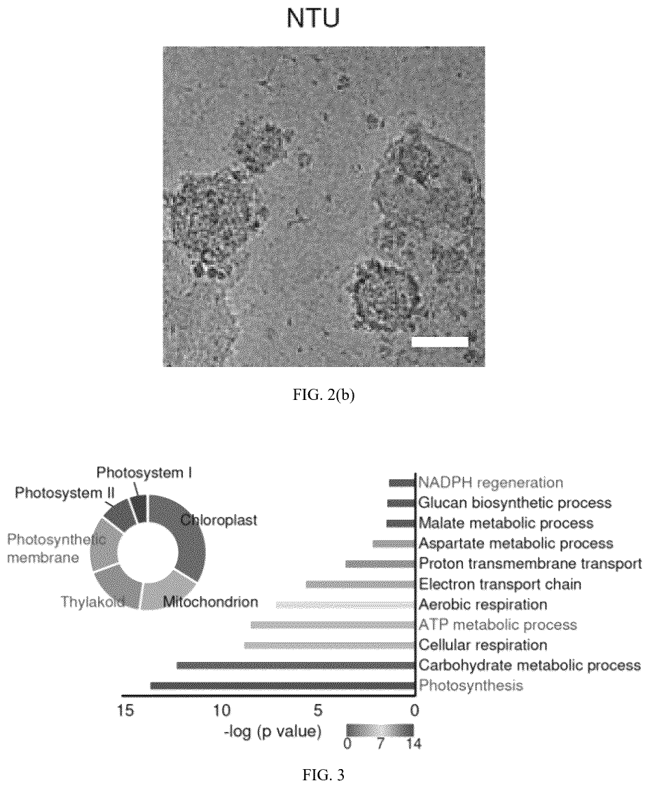

1. The extracted thylakoid and the nanocrystallized thylakoid were subjected to cryo-electron microscope sample preparation.
2. Cryo-electron microscope images of the thylakoid and the nano-thylakoid were photographed, indicating that the nano-thylakoid remained vesicular, as shown in FIG. 2(a) and FIG. 2(b).

### Protein Mass Spectrometry Analysis of Nano-Thylakoid in Embodiment 1

1. The nano-thylakoid was subjected to protein extraction, and then subjected to protein mass spectrometry analysis.
2. Results of the mass spectrometry analysis showed that the proteins related to ATP and NADPH generation were enriched, indicating that the nano-thylakoid retained the functional basis of ATP and NADPH generation, as shown in FIG. 3.

### Quantitative Measurement of ATP and NADPH Generation Capacities of Nano-Thylakoid in Embodiment 1

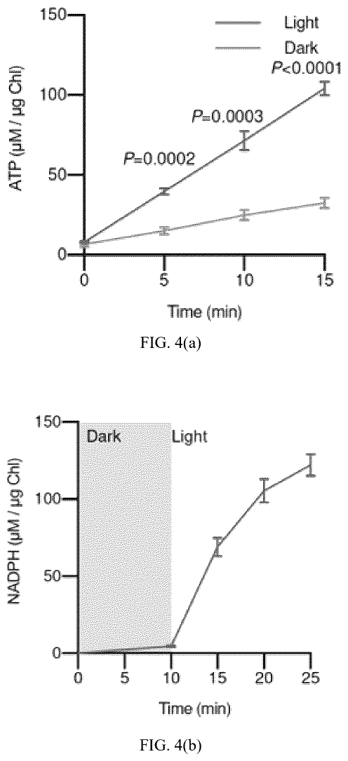

1. In a 0.7 mL reaction system (50 mM HEPES-KOH with pH of 7.8, 5 p72 M ferredoxin, 3 mM ADP, 5 mM K2HP04, 3 mM NADP⁺, 10 mM L-sodium ascorbate, 10 mM KCl, 5 mM MgCl₂, 1. 5 μM E. coli catalase, and 52 U mL−1 bovine superoxide dismutase), irradiation with ˜630 nm red light was carried out by an LED.
2. Samples were taken at 0 minute, 5^{th} minute, 10^{th} minute and 15^{th} minute, and an ATP concentration was measured by an ATP detection kit. For NADPH measurement, the samples were irradiated with red light for 10 minutes, and an NADPH yield was measured every 5 minutes by an NADPH detection kit.
3. Detection results showed that ATP and NADPH were significantly generated under the light condition, as shown in FIG. 4(a) and FIG. 4(b).

### Western Blotting of Half-Lives of Key Proteins of Nano-Thylakoid in Embodiment 1 Under Light and Dark Conditions

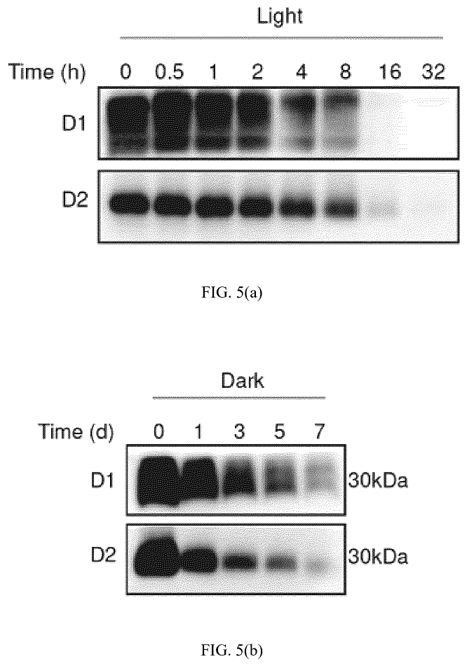

1. Abundance degrees of D1 and D2 proteins in NTU with passage of time under light and dark conditions were detected by protein blotting detection, and specifically, a Lysis Buffer was used to lyse the nano-thylakoid, and a WB western blotting experiment was carried out.
2. Bands were cut according to molecular weights of corresponding proteins, and a primary antibody and a secondary antibody were incubated respectively.
3. Changes of bands containing the D1 and D2 proteins could be seen after chemiluminescence exposure, the half-lives of the key proteins under the light condition were about 4 hours to 8 hours, and the half-lives of the key proteins under the dark condition were 3 days to 5 days, as shown in FIG. 5(a) and FIG. 5(b).

### Quantitative Measurement of Half-Lives of Functions of Nano-Thylakoid in Embodiment 1 Under Light and Dark Conditions

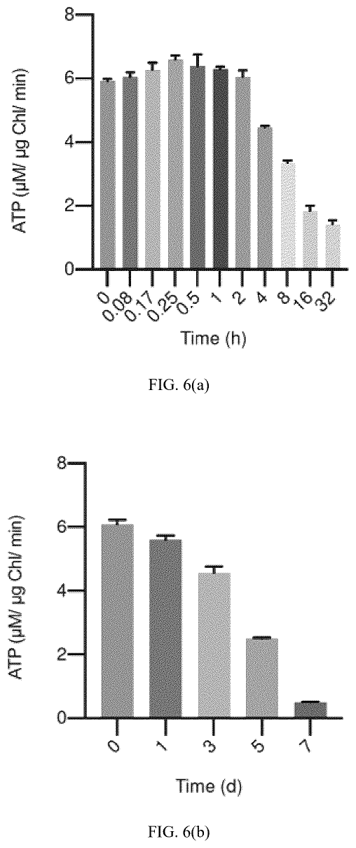

1. In a 0.7 mL reaction system (50 mM HEPES-KOH with pH of 7.8, 5 μM ferredoxin, 3 mM ADP, 5 mM K2HP04, 3 mM NADP*, 10 mM L-sodium ascorbate, 10 mM KCl, 5 mM MgCl₂, 1.5 μM E. coli catalase, and 52 U mL−1 bovine superoxide dismutase), irradiation with 630 nm red light was carried out by an LED.
2. Samples were taken at illustrated time, and ATP and NADPH concentrations were measured by ATP and NADPH detection kits.
3. Results showed that the half-lives of the functions under the light condition were about 4 hours to 8 hours, and the half-lives of the functions under the dark condition were about 3 days to 5 days, as shown in FIG. 6(a) and FIG. 6(b).

### Transmission Electron Microscope Characterization 0f Engineered Form of Nano-Thylakoid in Embodiment 1 Subjected to Liposome Encapsulation

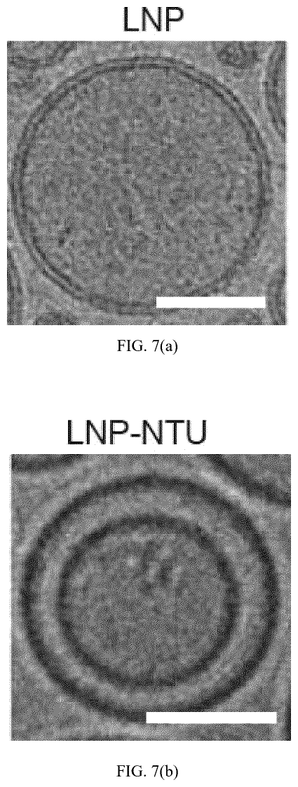

1. A liposome membrane and the nano-thylakoid were blended, and repeatedly extruded in the polycarbonate membrane with the pore size of 200 nm, so as to obtain a liposome-encapsulated engineered nano-thylakoid.
2. The extracted liposome-encapsulated engineered nano-thylakoid was subjected to cryo-electron microscope sample preparation.
3. Cryo-electron microscope images of the liposome-encapsulated engineered nano-thylakoid were photographed, indicating double-layer vesicle, which meant that the nano-thylakoid was successfully encapsulated into liposome vesicle, as shown in FIG. 7(a) and FIG. 7(b).

The nano-materials for generating ATP and NADPH under red light excitation obtained in Embodiments 2 and 3 were respectively subjected to particle size measurement, cryo-electron microscopy, protein mass spectrometry, quantitative measurement of ATP and NADPH generation capacities, western blotting of half-lives of key proteins under light and dark conditions, quantitative measurement of half-lives of functions under light and dark conditions, and transmission electron microscopy characterization of engineered form after liposome encapsulation, and the results were similar to those of the 100 nm nano-thylakoid prepared by ultrasonic extrusion nanocrystallization of the Spinacia oleracea leaf thylakoid in Embodiment 1, which indicated that the preparation of the nano-material for generating ATP and NADPH under red light excitation could be realized through other thylakoid sources, other thylakoid preparation schemes, other nanocrystallization methods and other target particle sizes of nanocrystallization mentioned above.

The above are only the preferred embodiments of the present invention, and it should be pointed out that, although the present invention has been described in detail with reference to the preferred embodiments above, those skilled in the art should understand that several improvements and decorations may also be made without departing from the principle of the present invention, and these improvements and decorations should also be regarded as falling within the scope of protection of the present invention without departing from the scope defined by the claims of the present invention.

We claim:

1. A nano-material for generating ATP and NADPH under red light excitation, wherein the nano-material is a nanocrystallized plant thylakoid; and a target particle size of the nanocrystallization is 50 nm to 200 nm.

2. The nano-material for generating ATP and NADPH under red light excitation according to claim 1, wherein the nanocrystallization is a granulation method, comprising heating, freeze-drying, extrusion and ultrasonic treatment.

3. The nano-material for generating ATP and NADPH under red light excitation according to claim 1, wherein an ultrasonic extrusion method is used for the nanocrystallization.

4. The nano-material for generating ATP and NADPH under red light excitation according to claim 1, wherein the particle size of the nanocrystallization is 100 nm.

5. The nano-material for generating ATP and NADPH under red light excitation according to claim 1, wherein the plant comprises a unicellular plant and a multicellular plant which contain the thylakoid or a similar photosynthetic structure.

6. The nano-material for generating ATP and NADPH under red light excitation according to claim 5, wherein the plant is a higher multicellular plant.

7. The nano-material for generating ATP and NADPH under red light excitation according to claim 6, wherein the higher multicellular plant is Spinacia oleracea and Arabidopsis thaliana.

8. A preparation method of the nano-material for generating ATP and NADPH under red light excitation according to claim 1, comprising the following steps of: extracting a plant thylakoid, and nanocrystallizing and granulating the plant thylakoid to obtain a nanocrystallized plant thylakoid.

9. The preparation method of the nano-material for generating ATP and NADPH under red light excitation according to claim 8, wherein the plant thylakoid is a Spinacia oleracea leaf thylakoid, and is extracted in the following mode:
in step 1: a Spinacia oleracea leaf material and a buffer A are mixed by a blender; a ratio of the Spinacia oleracea leaf material to the buffer A is 1:1 to 1:10 (w/v); ingredients of the buffer A are sorbitol, HEPES-KOH with pH of 7.6, MgCl₂ and 0.1% BSA; and a temperature of the buffer A is 4° C. to 10° C.;
in step 2: the obtained solution in the step 1 is filtered, and a filtrate is centrifuged at 3000 g for 10 minutes;
in step 3: the precipitate in the step 2 is resuspended in a buffer B; and ingredients of the buffer B are sorbitol, HEPES-KOH with pH of 7.6, MgCl₂, EDTA and L-sodium ascorbate; and
in step 4: the product in the step 3 is added into 80/40% Percoll gradient solution; and a part containing a green layer is collected to obtain the thylakoid.

10. The preparation method of the nano-material for generating ATP and NADPH under red light excitation according to claim 8, wherein the nanocrystallized granulation is ultrasonic extrusion nanocrystallization, and is specifically carried out in the following mode:
the thylakoid is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

11. The preparation method of the nano-material for generating ATP and NADPH under red light excitation according to claim 10, wherein conditions of the ultrasonic treatment comprise: a No. 2 amplitude-change pole, 20% to 60% power, turning on for 2 seconds, turning off for 3 seconds, and working for 2 minutes.

12. The preparation method of the nano-material for generating ATP and NADPH under red light excitation according to claim 10, wherein a pore size of the polycarbonate membrane above is 50 nm to 200 nm.
